(19) United States
(12) Patent Application Publication
(10) Pub. No.: US 2004/0006295 A1
(43) Pub. Date: Jan. 8, 2004

(54) ACL/MCL impact absorbtion device

(76) Inventors: Anthony S. Testa JR., Henderson, NV (US); Harry E. Elias, Lasvegas, NV (US); Robert J. Young, Las Vegas, NV (US)

Correspondence Address: ROBERT J YOUNG, 5025 S EASTERN AVENUE # 28, LAS VEGAS, NE, 89119, US

(21) Appl. No.: 10/189,228

(22) Filed: Jul. 5, 2002

(51) Int. Cl. A61F 5/00

(52) U.S. Cl. 602/26

(57) ABSTRACT

The central part to an ACL/MCL absorption device is a spring latterly attached to either side of an elastic knee brace. The springs extend the length of the lateral sides of the brace thus shielding the ACL/MCL ligaments from outside forces to prevent injuries to these ligaments.

### BACKGROUND OF THE INVENTION

1. Field of Invention

This invention is in the general field of sports protective devises, and more particularly, when worn, absorbs a percentage of the impact to the Anterior Cruciate Ligament (ACL) and Medial Collateral Ligament (MCL) surrounding the knee joint of the leg.

2. Description of the Prior Act

In the field of sports protective gear, many variations of an elasticized knee brace have been developed and marketed. These are worn to add support to an injured or injury recovering knee joint. None of the existing supports are built to absorb the impact upon the ACL/MCL ligaments to prevent the injury.

It should be appreciated that in the field of sports, the Anterior Cruciate Ligament (ACL) is the most commonly injured ligament of the knee.

### SUMMARY OF THE INVENTION

The object of the present invention is to provide a device to prevent injury by absorbing the impact to the ACL and MCL ligaments. The present invention takes the elasticized knee brace to a new stage by adding elasticized cylindrical pouches on either side of the brace, each containing a sturdy yet flexible spring which when worn, absorbs impact to both the ACL and MCL ligaments.

This invention addresses the Anterior Cruciate Ligament (ACL), Medial Collateral Ligament (MCL), to assist in preventing injuries, or at the least, reduce the severity of the injury and provide an impact support after an injury to allow the affected ligament to heal itself more easily. The existing knee braces assist after the injury and during periods of the healing process, but are not designed to relief the effects of continued impact aggravation to the affected area. Because of the rigidity yet flexibility of the springs, impact to the two ligaments is generally absorbed by the springs during a potential injury situation and thus prevents the injury and alleviates a need for periods of recovery or rehabilitation. In testing the device, because of the movement of the spring both from side to side and up and down, previously injured individuals found that the device assisted in alleviating pain the knee and ligament areas when walking and running. Individuals with arthritic conditions in this same area of the knee, found the device helpful in the same manner as the previously injured individuals. When walking or running, as the foot hits the ground, the knee joint absorbs the impact of that connection. The up and down movement of the springs within the device appears to lessen the impact of such mundane occurrences.

### BRIEF DISCRIPTION OF THE DRAWINGS

FIG. 1 is a perspective view of the preferred embodiment of the present invention;

FIG. 2 is a side view of the preferred embodiment of the present invention:

FIG. 3 is a side view of the protective device in the embodiment of FIG. 2;

FIG. 4 is a plain view of the protective device in the embodiment of FIG. 1;

FIG. 5 is a perspective view showing the interior makeup of the invention;

FIG. 6 is a side view showing the interior makeup of the invention.

### 3. DESCRIPTION OF THE PREFERRED EMBODIMENT

The invention provides a brace for the knee area of the leg and more particularly an impact absorption center to prevent injury to the ligaments surrounding the knee.

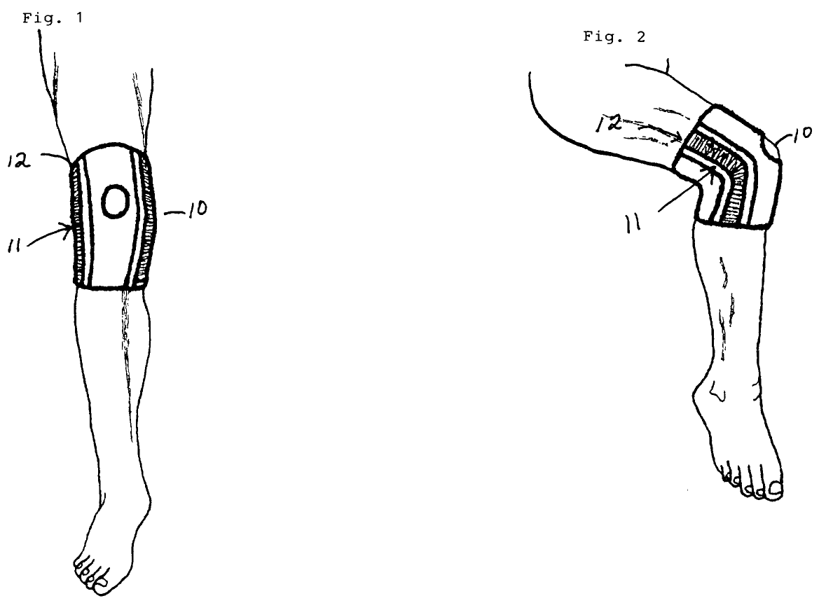

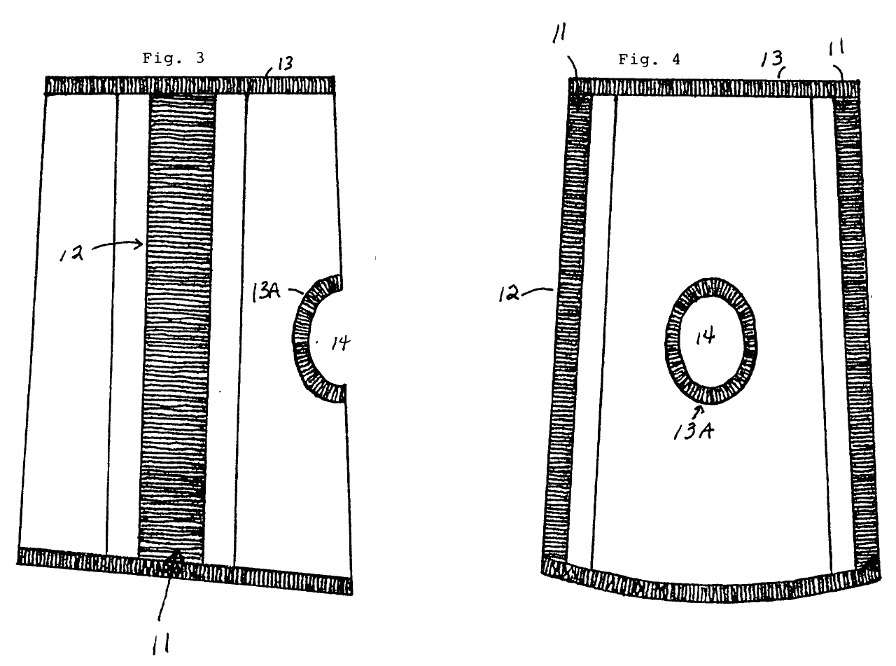

FIG. 1 is a frontal view of the device as worn. The brace 10 is worn over the knee joint of the human leg. A spring 11 is contained in an elasticized cylindrical pouch 12 on each outer side of the device. FIGS. 3, 4, 5,& 6, show the particular layout and construction of the device.

FIG. 2 is a side view of the device as worn. This view shows the spring's 11 location in juxtaposition to the device as it surrounds the knee.

As shown in FIG. 3, a 9/16″×8½″×0.054 mil spring 11 is inserted into a cylindrical pouch 12 or sleeve, which runs the entire length of the device. The pouch 12 consists of 1½″ heavy duty elastic sewn over and through ¾″ cotton web jute as a sleeve for the spring 11.

FIG. 4 shows a frontal view of the entire brace. The outer stitching 13 consists of ¼″ tight surge stitch over ten mil thick latex rubber sheeting to reinforce the fabric. The center hole 14 is surrounded by ¼ tight surge stitching 13A over ten mil thick latex rubber sheeting to reinforce the fabric.

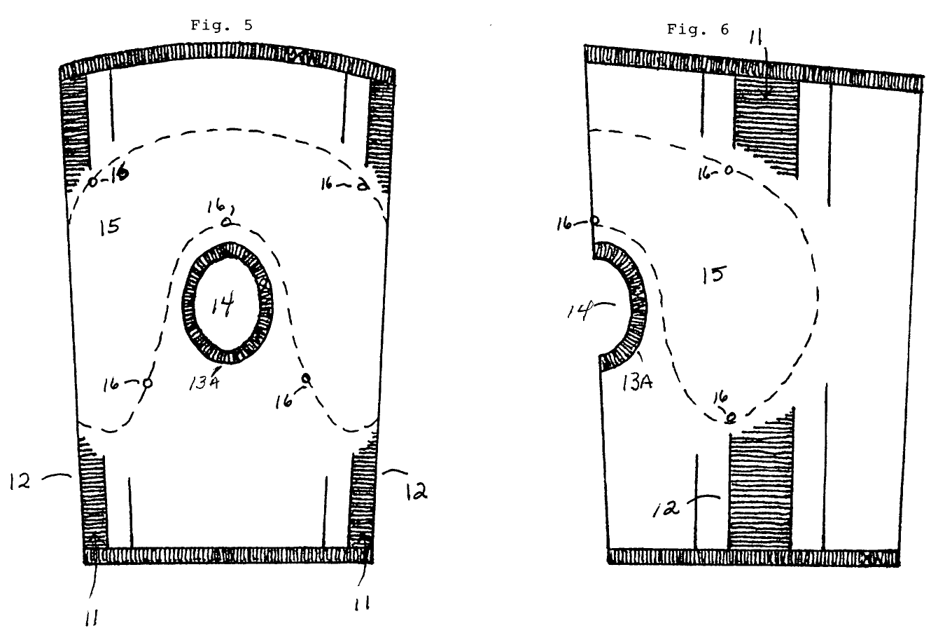

FIG. 5 is a frontal view of the brace showing a ⅜″ protective pad 15 covered with 100% cotton stretch fabric: stitched in place to the inside of the knee brace at stitch points 16.

FIG. 6 is a cross sectional view of FIG. 5 showing the location of the protective foam pad 15, and side view of stitch points 16.

While the invention has been particularly shown and described with reference to its preferred embodiments thereof, it should be understood that changes in length, width, and length of the spring may be made without departing from the spirit of the invention.

We claim:

1. By the introduction of the springs in the pouches on either side of a knee brace, as shown in FIG. 3 and FIG. 4 we have turned a simple knee brace into a device to absorb impact to the ACL/MCL ligaments. The rigidity of the springs act as a buffer between side impacts to the ligaments, while the flexibility of the spring allows it to move with the knee joint. In testing, we have found that with light to medium impact, the springs absorb so much of the impact energy that the leg moves only fractionally thus reducing injuries.

2. By the introduction of the springs into the common knee brace format, and since the length of the spring extends both above and below the knee cap, the up and down give and take of the springs absorb a portion of the impact to the knee area when walking or running. In testing, the device has been found helpful to individuals going through rehabilitation for knee injuries and individual with weak or arthritic problems in this same area. The rigidity of the springs supports the knee, while the flexibility of the springs absorbs impacts.
